(19) United States
(12) Patent Application Publication
(10) Pub. No.: US 2008/0194860 A1
(43) Pub. Date: Aug. 14, 2008

(54) NOVEL CRYSTAL OF (S)-(+)-2-(2-CHLOROPHENYL)-2-HYDROXY-ETHYL CARBAMATE

(75) Inventor: Michael Tauber, Brighton, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(73) Assignee: TRANSFORM PHARMACEUTICALS, INC., Lexington, MA (US)

(21) Appl. No.: 11/868,040

(22) Filed: Oct. 5, 2007

Related U.S. Application Data
(60) Provisional application No. 60/828,785, filed on Oct. 10, 2006.

(51) Int. Cl. C07C 271/18

(52) U.S. Cl. 560/29

(57) ABSTRACT

The present invention relates to a novel crystal of the Active Pharmaceutical Ingredient (API) (S)-(+)-2-(2-chlorophenyl)-2-hydroxy-ethyl carbamate, methods for the preparation of this crystal, pharmaceutical compositions comprising this crystal, and methods of treating a patient with this crystal.

### TECHNICAL FIELD

The present invention relates to a novel crystal of the Active Pharmaceutical Ingredient (API) (S)-(+)-2-(2-chlorophenyl)-2-hydroxy-ethyl carbamate, methods for the preparation of this crystal, pharmaceutical compositions comprising this crystal, and methods of treating a patient with this crystal.

### BACKGROUND OF THE INVENTION

The compound (S)-(+)-2-(2-chlorophenyl)-2-hydroxy-ethyl carbamate is an agent that can be used to treat a variety of disorders such as convulsions, epilepsy, stroke, muscle spasms, neuropathic pain, central nervous system disorders, and migraine. Its structure, properties, utility and preparation are described in U.S. Pat. no. 6,103,759, which is hereby incorporated by reference in its entirety.

Delivering an API to a patient requires more than just identifying a molecule and its use. An API must be formulated for delivery to a patient and this formulation (in addition to the API activity) is evaluated by regulatory agencies such as the US Food and Drug Administration (FDA) and the European Medicines Agency (EMEA). The FDA evaluates the formulation for, among other properties, delivery properties, stability, consistency, and manufacturing controls. An important factor in determining the properties of a particular formulation is the form of the API. APIs have been known to exist as amorphous forms, crystalline forms, polymorphs, hydrates and solvates. The forms for every API are different. While one particular API may be known to exist as a polymorph or a solvate, another API may be known to only exist in amorphous form. This form diversity is important because each different polymorph, solvate, hydrate or amorphous form may have different properties such as stability, solubility, and hygroscopicity.

Some forms of an API can be formulated into an FDA approvable formulation, while other forms lack the required properties to meet the high regulatory standards of the FDA. Even if a particular API can exist in more than one form suitable for formulation, different properties of an API form can affect the manufacturing process, shelf stability, route of administration, bioavailability and other important product characteristics. For example, the ability to improve or modulate stability or hygroscopicity can decrease manufacturing costs by reducing the need for humidity controlled chambers or reducing the need to package an API in humidity resistant packaging. In addition these same changes can increase product shelf stability thereby improving product distribution possibilities and affecting cost. In another example, one form of an API may have greater bioavailability than another form. Choosing the higher bioavailability form allows for a lower drug dose to be administered to a patient.

Thus, increasing the form diversity of a particular API increases opportunities to identify the ideal form for formulation. In addition, increasing form diversity increases the possibility of finding improved forms which can reduce manufacturing costs, increase shelf stability, offer new routes of administration, and offer new formulation options.

Applicants have discovered that (S)-(+)-2-(2-chlorophenyl)-2-hydroxy-ethyl carbamate can form a novel crystal possessing distinct physical properties and a distinct crystal structure different than previously known forms of (S)-(+)-2-(2-chlorophenyl)-2-hydroxy-ethyl carbamate. This discovery increases opportunities for the identification of an improved formulation suitable for FDA approval and for the ability of affect manufacturing process, shelf stability, route of administration, bioavailability and other product characteristics through crystal form selection.

### SUMMARY OF THE INVENTION

It has now been found that a novel crystal of (S)-(+)-2-(2-chlorophenyl)-2-hydroxy-ethyl carbamate can be obtained.

In one embodiment, the invention provides a Form α crystal with the chemical formula C₁₆ H₁₈ O₆S N Cl.

In another embodiment, the invention provides a Form α crystal with the chemical formula C₁₆ H₁₈ O₆ S N Cl and wherein said crystal comprises of (S)-(+)-2-(2-chlorophenyl)-2-hydroxy-ethyl carbamate.

In another embodiment, the invention provides a Form α crystal with the chemical formula C₁₆ H₁₈ O₆ S N Cl wherein said crystal is a co-crystal.

The invention also provides for methods of making the novel Form α crystal.

The invention also provides pharmaceutical compositions comprising this novel Form a crystal.

Compositions and methods of the invention are useful in the treatment or prevention of a variety of diseases including, among others, convulsions, epilepsy, stroke, muscle spasms, neuropathic pain, central nervous system disorders, and migraine.

### DESCRIPTION OF THE FIGURES

FIG. 1 illustrates powder X-ray diffraction (PXRD) measurements of a representative Form α crystal.

FIG. 2 illustrates differential scanning calorimetry (DSC) measurement of a representative Form β crystal.

FIG. 3 illustrates thermogravimetric analysis (TGA) measurements of a representative Form β crystal.

FIG. 4 is the molecular structure of the compound (S)-(+)-2-(2-chlorophenyl)-2-hydroxy-ethyl carbamate.

### DETAILED DESCRIPTION OF THE INVENTION

Applicants have discovered that (S)-(+)-2-(2-chlorophenyl)-2-hydroxy-ethyl carbamate can form a Form a crystal with the chemical formula C₁₆ H₁₈ 06 S N Cl. While Applicant's believe this Form α crystal is a co-crystal of (S)-(+)-2-(2-chlorophenyl)-2-hydroxy-ethyl carbamate and toluenesulfonic acid, it is possible that this Form α crystal is a tosylate salt of (S)-(+)-2-(2-chlorophenyl)-2-hydroxy-ethyl carbamate. Difficulties in analyzing the single crystal structure of this Form α crystal have prevented Applicant's from determining with absolute certainty whether the Form α crystal is a co-crystal or a salt. Regardless, Applicant's have isolated the Form α crystal, analyzed the Form α crystal with powder x-ray diffraction to identify the unique crystal pattern of this crystal, identified reproducible methods of making this Form α crystal.

The term “co-crystal” as used herein means a crystalline material comprised of two or more unique solids at room temperature (22 degrees C.), at least one of which is a co-crystal former. Solvates of (S)-(+)-2-(2-chlorophenyl)-2-hydroxy-ethyl carbamate that do not further comprise a co-crystal former are not co-crystals according to the present invention. The co-crystals may however, include one or more solvate molecules in the crystalline lattice. An API bound to an acid or base in the form of a salt can be one unique solid, but it cannot be two unique solids by itself.

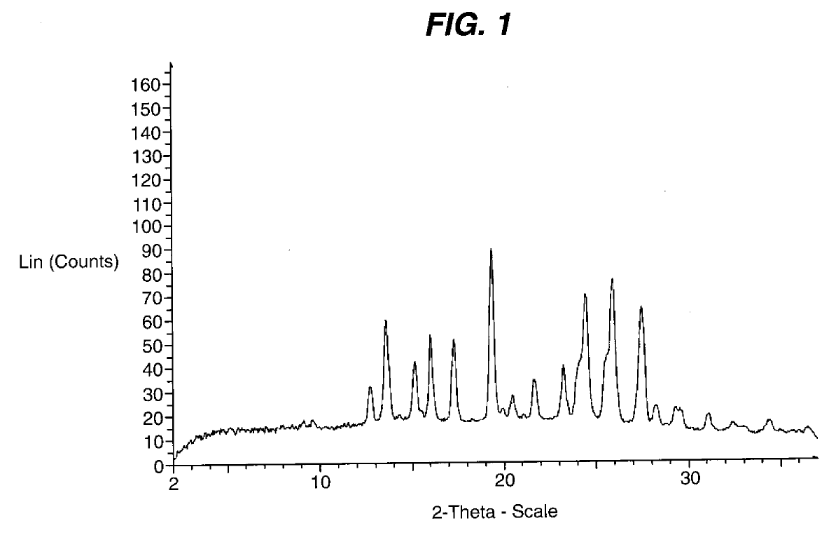

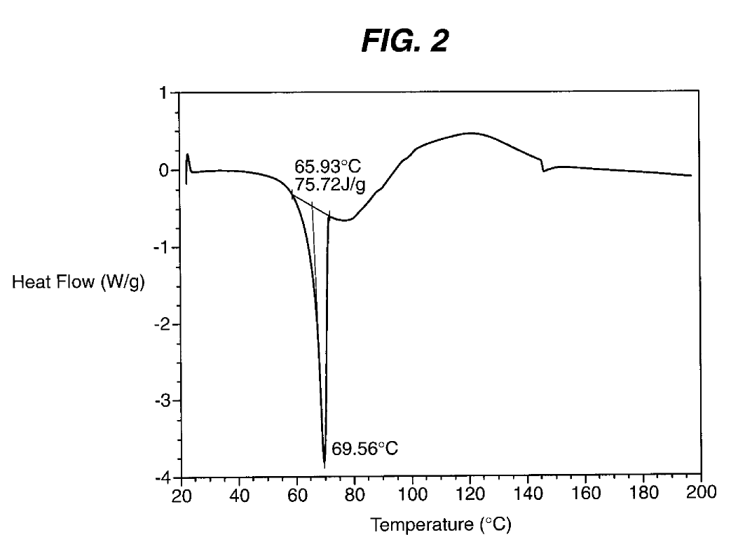

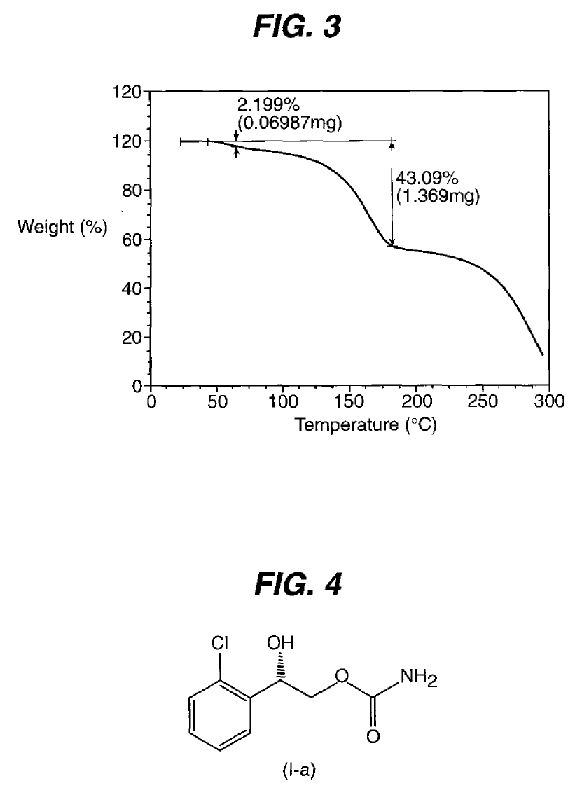

In one embodiment, the invention provides a Form α crystal with the chemical formula C₁₆ H₁₈ O₆ S N Cl. In one aspect of this invention, a Form α crystal is characterized by a powder X-ray diffraction pattern having one powder X-ray diffraction peak at about 13.6 degrees 2-theta. In another aspect of this invention, a Form α crystal is characterized by a powder X-ray diffraction pattern having powder X-ray diffraction peaks at about 13.6 and 16.0 degrees 2-theta. In one aspect of this invention, a Form α crystal is characterized by a powder X-ray diffraction pattern having powder X-ray diffraction peaks at about 13.6, 16.0, and 25.9 degrees 2-theta. In a further aspect of this invention, a Form α crystal is characterized by a powder X-ray diffraction pattern having powder X-ray diffraction peaks at about 12.7, 13.6, 15.1, 16.0 and 25.9 degrees 2-theta. In a still further aspect of this invention, a Form α crystal is characterized by a powder X-ray diffraction pattern having powder X-ray diffraction peaks at about 12.7, 13.6, 15.1, 16.0, 17.3 and 25.9 degrees 2-theta. In another aspect of this invention, a Form α crystal is characterized by a powder X-ray diffraction pattern having powder X-ray diffraction peaks at about 12.7, 13.6, 15.1, 16.0, 17.3, 21.6 and 25.9 degrees 2-theta. In one aspect of this invention, a Form α crystal is characterized by a powder X-ray diffraction pattern that is substantially similar to the powder X-ray diffraction pattern of FIG. 1. In one aspect of this invention, a Form α crystal is characterized by a TGA thermogram comprising about 43% percent weight loss between about 25 degrees C. and about 182 degrees C. In another aspect of this invention, a Form α crystal is characterized by a TGA thermogram substantially similar to the TGA thermogram in FIG. 3. In still another aspect of this invention, a Form α crystal is characterized by an endothermic transition at about 69 degrees C. In a further aspect of this invention, a Form α crystal is characterized by a differential scanning calorimetry (DSC) measurement substantially similar to the DSC in FIG. 2. In one aspect of this invention, a Form α crystal is substantially pure. In another aspect of this invention, a Form α crystal is a co-crystal.

In another embodiment, the invention provides a Form α crystal with the chemical formula C₁₆ H₁₈ O₆ S N Cl wherein said crystal comprises (S)-(+)-2-(2-chlorophenyl)-2-hydroxy-ethyl carbamate. In a further embodiment, the invention provides a Form α crystal with the chemical formula C₂₁ H₂₄ F N₅ O₇ wherein said crystal comprises (S)-(+)-2-(2-chlorophenyl)-2-hydroxy-ethyl carbamate and toluenesulfonic acid. In a still further embodiment, the invention provides a Form α crystal with the chemical formula C₂₁ H₂₄ F N₅ O₇ wherein said crystal comprises (S)-(+)-2-(2-chlorophenyl)-2-hydroxy-ethyl carbamate and p-toluenesulfonic acid. In a further embodiment, the invention provides for pharmaceutical compositions comprising a Form α crystal with the chemical formula C₁₆ H₁₈ O₆ S N Cl.

In one embodiment, the invention provides for a crystal with the chemical formula C₁₆ H₁₈ O₆ S N Cl, wherein said crystal is characterized by a powder X-ray diffraction pattern having one powder X-ray diffraction peak at about 4.3 degrees 2-theta. In another embodiment, the invention provides for a crystal with the chemical formula C₁₆ H₁₈ O₆ S N Cl, wherein said crystal is characterized by a powder X-ray diffraction pattern having powder X-ray diffraction peaks at about 4.3 and 11.7 degrees 2-theta. In a further embodiment, the invention provides for a crystal with the chemical formula C₁₆ H₁₈ O₆ S N Cl, wherein said crystal is characterized by a powder X-ray diffraction pattern having powder X-ray diffraction peaks at about 4.3, 11.7, and 16.3 degrees 2-theta. In a still further embodiment, the invention provides for a crystal with the chemical formula C₁₆ H₁₈ O₆ S N Cl, wherein said crystal is characterized by a powder X-ray diffraction pattern having powder X-ray diffraction peaks at about 4.3, 8.9, 11.7, 15.6, and 16.3 degrees 2-theta. In another embodiment, the invention provides for a crystal with the chemical formula C₁₆ H₁₈ O₆ S N Cl, wherein said crystal is characterized by a powder X-ray diffraction pattern having powder X-ray diffraction peaks at about 4.3, 8.9, 11.7, 15.6, 16.3, and 17.9 degrees 2-theta. In a further embodiment, the invention provides for a crystal with the chemical formula C₁₆ H₁₈ O₆ S N Cl, wherein said crystal is characterized by a powder X-ray diffraction pattern having powder X-ray diffraction peaks at about 4.3, 8.9, 11.7, 15.6, 16.3, 17.9, 19.1, and 22.6 degrees 2-theta.

It has been found that a Form α crystal with the chemical formula C₁₆ H₁₈ O₆ S N Cl has improved or different properties than compared to prior known forms of (S)-(+)-2-(2-chlorophenyl)-2-hydroxy-ethyl carbamate. In particular, Form α has a distinct crystal structure and a distinct chemical composition.

Compositions and methods of the invention are useful in the treatment or prevention of a variety of diseases including, among others, bacterial infections, fungal infections, and infectious disease.

Assaying the solid phase for the presence of a Form α crystal may be carried out by conventional methods known in the art. For example, X-ray diffraction techniques can be used to assess the presence of crystals. Other techniques, used in an analogous fashion, include differential scanning calorimetry (DSC), thermogravimetric analysis (TGA), infrared spectroscopy (IR), single crystal X-ray diffraction and Raman spectroscopy. FIG. 1 shows PXRD measurements of representative Form α crystals. FIG. 2 shows DSC measurements of representative Form α crystals. FIG. 3 shows TGA measurements of representative Form α crystals.

In one embodiment, the invention provides for a method of making a Form α crystal with the chemical formula C₁₆ H₁₈ O₆ S N Cl comprising the steps of cocrystallizing (S)-(+)-2-(2-chlorophenyl)-2-hydroxy-ethyl carbamate with toluenesulfonic acid and isolating the crystal. In another embodiment, the use of an excess (more than 1 molar equivalent for a 1:1 toluenesulfonic acid) of toluenesulfonic acid can be used to drive the formation of a Form α crystal. Such an excessive use of toluenesulfonic acid to form a crystal can be employed in solution or when grinding (S)-(+)-2-(2-chlorophenyl)-2-hydroxy-ethyl carbamate and toluenesulfonic acid to cause Form α crystal formation.

The Form α crystal obtained as a result of such process steps may be readily incorporated into a pharmaceutical composition (or medicament) by conventional means. Pharmaceutical compositions and medicaments may further comprise a pharmaceutically-acceptable diluent, excipient or carrier. In one embodiment, the Form α crystal and formulations comprising (S)-(+)-2-(2-chlorophenyl)-2-hydroxy-ethyl carbamate, are suitably stable for pharmaceutical use.

For preparing pharmaceutical compositions from the Form α crystal described by this invention, inert, pharmaceutically acceptable carriers can be either solid or liquid. Solid form preparations include powders, tablets, dispersible granules, capsules, cachets and suppositories. Tablets, powders, cachets and capsules can be used as solid dosage forms suitable for oral administration. Examples of pharmaceutically acceptable carriers and methods of manufacture for various compositions may be found in A. Gennaro (ed.), The Science and Practice of Pharmacy, 20.sup.th Edition, Lippincott Williams & Wilkins, Baltimore, Md., (2000).

Liquid form preparations include solutions, suspensions and emulsions. Aerosol preparations suitable for inhalation may include solutions and solids in powder form, which may be in combination with a pharmaceutically acceptable carrier, such as an inert compressed gas, e.g., nitrogen. Also included are solid form preparations that are intended to be converted, shortly before use, to liquid form preparations for either oral or parenteral administration. Such liquid forms include solutions, suspensions and emulsions.

Specific dosage and treatment regimens for any particular patient may be varied and will depend upon a variety of factors, the age, body weight, general health status, sex and diet of the patient, the time of administration, the rate of excretion, the specific drug combination, the severity and course of the symptoms being treated, the patient's disposition to the condition being treated and the judgment of the treating physician. Determination of the proper dosage regimen for a particular situation is within the skill of the art. The amount and frequency of the administration of the compositions of this invention, or the pharmaceutical compositions thereof, may be regulated according to the judgment of the attending clinician, based on the factors recited above. As a skilled artisan will appreciate, lower or higher doses than those recited above may be required.

The Crystal of the Present Invention was Analyzed Using the Following Methods.

Powder x-ray diffraction patterns were obtained using either a D/Max Rapid X-ray Diffractometer (Rigaku/MSC, The Woodlands, TX, U.S.A.) or a Bruker D8 Discover with GADDS diffractometer (Bruker-AXS Inc., Madison, Wis., U.S.A).

The D/Max Rapid X-ray Diffractometer was equipped with a copper source (Cu/K_{α}1.5406 Å), manual x-y stage, and 0.3 mm collimator. A sample was loaded into a 0.3 mm quartz capillary tube (Charles Supper Company, Natick, Mass., U.S.A.) by sectioning off the closed end of the tube and tapping the small, open end of the capillary tube into a bed of the powdered sample or into the sediment of a slurried sample. The loaded capillary tube was mounted in a holder that was placed and fitted into the x-y stage. A diffractogram was acquired using control software (RINT Rapid Control Software, Rigaku Rapid/XRD, version 1.0.0 (©1999 Rigaku Co.)) under ambient conditions at a power setting of 46 kV at 40 mA in transmission mode, while oscillating about the omega-axis from 0-5 degrees at 1 degree/second, and spinning about the phi-axis over 360 degrees at 2 degrees/second. The exposure time was 15 minutes unless otherwise specified.

The diffractogram obtained was integrated of 2-theta from 2-40 degrees and chi (1 segment) from 0-36 degrees at a step size of 0.02 degrees using the cyllnt utility in the RINT Rapid display software (RINT Rapid display software, version 1.18 (Rigaku/MSC)) provided by Rigaku with the instrument. The dark counts value was set to 8 as per the system calibration by Rigaku. No normalization or omega, chi, or phi offsets were used for the integration.

The Bruker D8 Discover with GADDS Diffractometer was equipped with a copper source (Cu/K_{α}1.5406 Å), computer controlled x-y-z stage, a 0.5 mm collimator and a Hi-Star area detector. Samples were loaded into a proprietary sample holder by tapping the sample holder into a powder bed and arraying the holders into a 96 position block. The block was then loaded onto the x-y-z stage and the sample positions were entered into the software. A diffractogram was acquired using control software (GADDS—General Area Detector Diffraction System, (Bruker, version 4.1.14 (©1997-2003 Bruker-AXS.)) under ambient conditions at a power setting of 46 kV at 40 mA in reflectance mode. The exposure time was 5 minutes unless otherwise specified.

The diffractogram obtained was integrated of 2-theta from 2-40 degrees and chi (1 segment) from 0-36 degrees at a step size of 0.02 degrees using the GADDS software.

The relative intensity of peaks in a diffractogram is not necessarily a limitation of the PXRD pattern because peak intensity can vary from sample to sample, e.g., due to crystalline impurities. Further, the angles of each peak can vary by about +/−0.1 degrees, or by about +/−0.05. The entire pattern or most of the pattern peaks may also shift by about +/−0.1 degrees to about +/−0.2 degrees due to differences in calibration, settings, and other variations from instrument to instrument and from operator to operator. All reported PXRD peaks in the Figures, Examples, and elsewhere herein are reported with an error of about ±0.1 degrees 2-theta. Unless otherwise noted, all diffractograms are obtained at about room temperature (about 24 degrees C. to about 25 degrees C.).

For PXRD data herein, including Tables and Figures, each composition of the present invention may be characterized by any one, any two, any three, any four, any five, any six, any seven, or any eight or more of the 2 theta angle peaks.

The following specific examples illustrate the present invention in more detail. They are, however, not intended to limit its scope in any manner.

### EXAMPLES

### Example 1

### Cocrystalization of a Form α Crystal

15 mg (S)-(+)-2-(2-chlorophenyl)-2-hydroxy-ethyl carbamate and 11.9 mg p-toluenesulfonic acid monohydrate were ground in a ball mill for 10 min. The resulting solid was analyzed by powder X-ray diffraction.

### Example 2

### Cocrystalization of a Form α Crystal

15 mg (S)-(+)-2-(2-chlorophenyl)-2-hydroxy-ethyl carbamate, 11.9 mg p-toluenesulfonic acid monohydrate, and 10 ul of hexane were ground in a ball mill for 10 min. The resulting solid was analyzed by powder X-ray diffraction.

### Example 3

### Cocrystalization of a Form α Crystal

30.2 mg (S)-(+)-2-(2-chlorophenyl)-2-hydroxy-ethyl carbamate, 24.4 mg p-toluenesulfonic acid monohydrate (1:1 molar ratio) and 10 ul hexane were ground in a steal wig-l-bug along with a grinding ball for 10 minutes. The sample was allowed to equilibrate overnight before analysis. The resulting solid was analyzed by powder X-ray diffraction.

1. A Form a crystal with the chemical formula C₁₆ H₁₈ O₆ S N Cl.

2. The crystal of claim 1, wherein said crystal is characterized by a powder X-ray diffraction pattern having one powder X-ray diffraction peak at about 13.6 degrees 2-theta.

3. The crystal of claim 1, wherein said crystal is characterized by a powder X-ray diffraction pattern having powder X-ray diffraction peaks at about 13.6 and 16.0 degrees 2-theta.

4. The crystal of claim 1, wherein said crystal is characterized by a powder X-ray diffraction pattern having powder X-ray diffraction peaks at about 13.6, 16.0, and 25.9 degrees 2-theta.

5. The crystal of claim 1, wherein said crystal is characterized by a powder X-ray diffraction pattern having powder X-ray diffraction peaks at about 12.7, 13.6, 15.1, 16.0 and 25.9 degrees 2-theta.

6. The crystal of claim 1, wherein said crystal is characterized by a powder X-ray diffraction pattern having powder X-ray diffraction peaks at about 12.7, 13.6, 15.1, 16.0, 17.3 and 25.9 degrees 2-theta.

7. A crystal with the chemical formula C₂₁ H₂₄ F N₅ O₇, wherein said crystal is characterized by a powder X-ray diffraction pattern that is substantially similar to the powder X-ray diffraction pattern of FIG. 1.

8. The crystal of claim 1, wherein said crystal is a co-crystal.

9. A crystal with the chemical formula C₁₆ H₁₈ O₆ S N Cl, wherein said crystal is characterized by a powder X-ray diffraction pattern having one powder X-ray diffraction peak at about 13.6 degrees 2-theta.

10. The crystal of claim 9, wherein said crystal is characterized by a powder X-ray diffraction pattern having powder X-ray diffraction peaks at about 13.6 and 16.0 degrees 2-theta.

11. The crystal of claim 9, wherein said crystal is characterized by a powder X-ray diffraction pattern having powder X-ray diffraction peaks at about 13.6, 16.0, and 25.9 degrees 2-theta.

12. The crystal of claim 9, wherein said crystal is characterized by a powder X-ray diffraction pattern having powder X-ray diffraction peaks at about 12.7, 13.6, 15.1, 16.0 and 25.9 degrees 2-theta.

13. The crystal of claim 9, wherein said crystal is characterized by a powder X-ray diffraction pattern having powder X-ray diffraction peaks at about 12.7, 13.6, 15.1, 16.0, 17.3 and 25.9 degrees 2-theta.

14. The crystal of claim 9, wherein said crystal is characterized by a powder X-ray diffraction pattern having powder X-ray diffraction peaks at about 12.7, 13.6, 15.1, 16.0, 17.3, 21.6 and 25.9 degrees 2-theta.

15. A crystal with the chemical formula C₁₆ H₁₈ O₆ S N Cl, wherein said crystal comprises (S)-(+)-2-(2-chlorophenyl)-2-hydroxy-ethyl carbamate.

16. The crystal of claim 15, wherein said crystal comprises toluenesulfonic acid.

17. The crystal of claim 15, wherein said crystal is characterized by a powder X-ray diffraction pattern having powder X-ray diffraction peaks at about 13.6 and 16.0 degrees 2-theta.

18. The crystal of claim 15, wherein said crystal is characterized by a powder X-ray diffraction pattern having powder X-ray diffraction peaks at about 13.6, 16.0, and 25.9 degrees 2-theta.

19. The crystal of claim 15, wherein said crystal is characterized by a powder X-ray diffraction pattern having powder X-ray diffraction peaks at about 12.7, 13.6, 15.1, 16.0 and 25.9 degrees 2-theta.

20. The crystal of claim 15, wherein said crystal is characterized by a powder X-ray diffraction pattern having powder X-ray diffraction peaks at about 12.7, 13.6, 15.1, 16.0, 17.3 and 25.9 degrees 2-theta.

21. The crystal of claim 15, wherein said crystal is characterized by a powder X-ray diffraction pattern having powder X-ray diffraction peaks at about 12.7, 13.6, 15.1, 16.0, 17.3, 21.6 and 25.9 degrees 2-theta.

22. A method of making a crystal with the chemical formula C₁₆ H₁₈ O₆ S N Cl, comprising the steps of cocrystallizing (S)-(+)-2-(2-chlorophenyl)-2-hydroxy-ethyl carbamate with toluenesulfonic acid and isolating the crystal.

23. A pharmaceutical composition comprising the crystal of claims 1, 9 or 15.

24. A crystal obtained by the cocrystallization of (S)-(+)-2-(2-chlorophenyl)-2-hydroxy-ethyl carbamate with toluenesulfonic acid.

25. A pharmaceutical composition comprising the crystal of claim 24.
